(19) United States
(12) Patent Application Publication
(10) Pub. No.: US 2007/0053918 A1
(43) Pub. Date: Mar. 8, 2007

(54) Injectable depots consisting of liposomal aggregates for the delivery of active substances

(75) Inventors: Steffen Panzner, Halle (DE); Silke Lutz, Halle (DE)

Correspondence Address: NORRIS, MCLAUGHLIN & MARCUS, P.A., 875 THIRD AVE, 18TH FLOOR, NEW YORK, NY, 10022, US

(73) Assignee: NOVOSOM AG, HALLE (DE)

(21) Appl. No.: 10/556,703

(22) PCT Filed: May 10, 2004
(86) PCT No.: PCT/DE04/01020

§ 371 Date: Nov. 10, 2005

(30) Foreign Application Priority Data
May 12, 2003 (DE) 103 22 123.9
May 10, 2004 (DE) 10 2001 017 996.4

(51) Int. Cl. A61K 38/28; A61K 31/727; A61K 39/00; A61K 9/127

(52) U.S. Cl. 424/184.1; 424/450; 514/15; 514/56; 514/3

(57) ABSTRACT

The invention relates to formulations of liposomes and polymers for the production of an injectable depot of active substances, which has sustained release and effect in a mammal organism.

The invention relates to the use of a depot system for the delayed release of active substances in basic research and medicine.

Following application, peptide and protein active substances undergo very rapid degradation in the body or elimination and therefore must be administered by repeated injections. To increase the “patient compliance”, a suitable delivery system is required which protects the active substance from degradation in the body, gradually releasing it into the bloodstream. Depot systems being injected subcutaneously or intramuscularly are used as such delivery systems. Liposomes are one possible form of such a carrier system. They are constituted of one or more lipid double layers, enclosing in their inside an aqueous compartment allowing entrapment of water-soluble substances. The lipid double layer allows incorporation of lipophilic substances.

According to the state of the art, liposomes composed of neutral, anionic or PEG lipids are used for depot systems, e.g. in WO 9920301 for a depot of γ-interferon, in Diabetes 31 (1982), 506-511, for a depot of insulin; furthermore, in Proc. Natl. Acad. Sci. 88 (1991) for vaccination.

In BBA 1328 (1997), 261-272, various liposomal systems (unilamellar and multilamellar) of egg PC, egg PG, DPPC, DPPG, PS and cholesterol have been investigated for their reception in the lymphatic system and their biodistribution following subcutaneous administration. The review article Advanced Drug Delivery Reviews 50 (2001), 143-156, represents a continuation of the above investigations, demonstrating that liposomes smaller in size (<150 nm) migrate from a subcutaneous depot into the lymph.

According to the state of the art, neutral and negatively charged liposomes have been used in liposomal depot systems. For migration into the lymph to be absent, the liposomes must have a minimum size.

Other publications refer to such size dependence, and small liposomes are encapsulated in various matrices, by means of which migration is prevented. Such basic structures may consist of synthetic polymers (Bos et al., Biopharm Europe, November 2001, 64-74; Bezemer et al., J. Controlled Release 62 (1999), 393-405; Stenekes et al., Pharm. Res. 17 (2000), 690-695) or may utilize natural structures such as a fibrin network (Meyenburg et al., J. Controlled Release 69 (2000), 159-168). p= Frequently, however, such polymers, particularly under in vivo conditions, have a disadvantageous effect on the stability of liposomes. Thus, Meyenburg et al. have achieved a half-life as short as a few days which is not substantially above that of the free active substance.

Another approach to a controllable liberation of active substances enclosed in liposomes has been described in the publication by Cullis et al. (BBA 1565 (2002), 129-135). Doxorubicin has been entrapped in anionic liposomes and contacted with cationic liposomes, thereby rapidly releasing the active substance. By modifying the liposome composition, it has been possible to extend the active substance release phase in an in vitro test array to several hours. However, a large number of uses require monthly depots in vivo, which, however, could not be provided as yet.

The object of the invention was therefore to provide new stable liposomal depot formulations which would achieve sustained release of an active substance for at least one week and have good tolerability in an organism, particularly in a mammal organism.

The invention solves the above technical problem by using a depot system for delayed release of active substances, comprising anionic liposomes with (a) saturated synthetic phosphatidyl cholines selected from the group of DMPC, DPPC and/or DSPC, (b) cholesterol with a percentage of from 35 to 50 mole-%, (c) anionic lipids selected from the group of DMPG, DPPG, DSPG, DMPS, DPPS and/or CHEMS with a percentage of from 5 to 20 mole-% in the liposomal membrane, and at least one protein and/or peptide active substance and a cationic polymer. By using this formulation of active substances, the latter are present in the form of aggregates, particularly when used as a depot. Such liposomal aggregates are constituted of anionic liposomes and cationic polymers.

The lipid composition of the invention includes saturated backbone lipids and cholesterol which provide integrity of the liposomes even in the aggregated state, thus advantageously resulting in improved protection of the active substance, as well as longer depot times. As a result of their interaction with the cationic polymers, the anionic lipids advantageously cause formation of aggregates which, owing to their size and charge, or as a result of aggregation with serum components and components of the interstitial fluid, remain at the site of puncture and do not migrate into the lymph, for example.

Advantageously, the liposomal aggregates according to the invention have a depot time of at least one week, preferably more than 10 to 14 days, and more preferably longer than 3 weeks.

Advantageously, the production process can be performed without organic, water-immiscible solvents possibly causing regulatory problems because complete removal thereof is difficult or damage is done to the active substance, e.g. proteins.

In a particularly advantageous embodiment, cationic liposomes are used as aggregate-forming polymers. The aggregates can be formed during production to be ready when applied. However, it is also possible to mix the two components as a solution or suspension shortly prior to or immediately during use. The production of the liposomal containers and the formation of larger aggregates are two process steps, and each of these steps advantageously can be designed in such a way that the respective solutions or suspensions allow sterile filtration. In this way, said larger aggregates, by means of which diffusion away from the puncture point is avoided, are formed in a subsequent and very uncomplicated process step.

Methods of entrapping water-soluble protein and/or peptide active substances in liposomes are well-known to those skilled in the art: extrusion through polycarbonate membranes, ethanol injection or high pressure homogenization.

In another preferred embodiment of the present invention, liposomes constituted of neutral and anionic lipids are used as liposomal depot system for the delayed release of therapeutic peptides and proteins of a wide variety of molar masses. J. Pharm. Sci. 89(3), 297-310, 2000, discloses the absolute bioavailabilities of peptides and proteins of varying size following subcutaneous application, wherein no significant reduction in bioavailability with increasing molar mass has been observed. Depot systems for membrane proteins do not represent a subject matter of the teaching according to the invention.

Therapeutic peptides and proteins undergo very rapid degradation in the body, for which reason they must be administered by repeated injections. The peptides and proteins, analogs thereof, related peptides, fragments, inhibitors and antagonists relevant to this embodiment of the invention comprise:

Transforming growth factors (TGF-alpha, TGF-beta), interleukins (e.g. IL-1, IL-2, IL-3), interferons (IFN-alpha, IFN-beta, IFN-gamma), calcitonin, insulin-like growth factors (IGF-1, IGF-2), parathyroid hormone, granulocyte colony-stimulating factor (GCSF), granulocyte macrophage colony-stimulating factor (GMCSF), macrophage colony-stimulating factor (MCSF), erythropoietin, insulins, amylins, glucagons, lipocortins, growth hormones, somatostatin, angiostatin, endostatin, octreotide, gonadotropin-releasing hormone (GNRH), luteinizing hormone-releasing hormone (LHRH), and effective agonists such as leuprolide acetate, buserelin, goserelin, triptorelin; platelet-derived growth factor; blood-clotting factors (e.g. factor VIII, factor IX), thromboplastin activators, tissue plasminogen activators, streptokinase, vasopressin, muramyl dipeptides (MDP), atrial natriuretic factor (ANF), calcitonin gene-related peptide (CGRP), bombesin, enkephalins, enfuvirtides, vasoactive intestinal peptide (VIP), epidermal growth factor (EGF), fibroblast growth factor (FGF), growth hormone-releasing hormone (GRH), bone morphogenetic proteins (BMP), antibodies and antibody fragments (e.g. scFv fragments, Fab fragments), peptide T and peptide T amides, herpes virus inhibitor, virus replication inhibition factor, antigens and antigen fragments, soluble CD4, ACTH and fragments, angiotensins, and ACE inhibitors, bradykinin (BK), hypercalcemia malignancy factor (PTH-like adenylate cyclase-stimulating protein), beta-casomorphins, chemotactic peptides and inhibitors, corticotropin-releasing factor (CRF), caerulein, cholecystokinins+fragments and analogs, galanin, gastric inhibitory polypeptide (GIP), gastrins, gastrin-releasing peptide (GRP), motilin, PHI peptides, PHM peptides, peptide YY, secretins, melanocyte-stimulating hormone (MSH), neuropeptide Y (NPY), neuromedins, neuropeptide K, neurotensins, phosphate acceptor peptide (c-AMP protein kinase substrates), oxytocins, substance P, TRH, as well as fragments, analogs and derivatives of the above substances.

In another preferred embodiment, water-soluble active substances or water-soluble derivatives of active substances from the following classes of active substances are used:
antibiotics, preferably rifamycin SV Na salt, rifampicin, tetracycline hydrochloride, kanamycin, penicillin G, ampicillin and/or novobiocin,
antimycotic agents, preferably amphotericin B and/or flucytosine,
cytostatic agents, preferably doxorubicin, daunorubicin, vincristine and/or cytarabin,
glucocorticoids, preferably dexamethasone, prednisolone, hydrocortisone and/or betamethasone.

In addition to the above-mentioned classes of active substances, carbohydrates such as heparin or hyaluronic acid can be active substance molecules relevant to this invention. Membrane proteins, being difficult to introduce in the inner space of liposomes, do not represent preferred active substances in the meaning of the invention.

Membrane-forming and membranous lipids are possible as lipids for the active substance-loaded liposomes, and they can be of natural or synthetic origin. More specifically, these include cholesterol and derivatives, phosphatidyl cholines, phosphatidyl ethanolamines as neutral lipids. In a particularly preferred fashion, completely saturated compounds from this class are used, for example:
dimyristoylphosphatidyl choline (DMPC)
dipalmitoylphosphatidyl choline (DPPC) and/or
distearoylphosphatidyl choline (DSPC).

Preferred anionic lipids in the practice of the invention are cholesterol hemisuccinate (CHEMS), phosphatidyl glycerols, phosphatidyl serines and phosphatidic acids. In addition, other membrane-forming or membranous substances with a negative charge, such as alkylcarboxylic acids or dialkyl phosphates with alkyl chains between 16 and 20 C atoms, can be incorporated in the liposomal bilayer.

In a particularly preferred composition, saturated synthetic phosphatidyl cholines such as DMPC, DPPC or DSPC, cholesterol and the anionic lipids DMPG, DPPG, DSPG or DMPS, DPPS or CHEMS are used, and in an even more preferred fashion the percentage of anionic lipids in the liposome membrane is between 5 and 20 mole-%, and the percentage of cholesterol is between 35 and 50 mole-%.

The size of the liposomes varies from 20 to 1000 nm, particularly from 50 to 800 nm, preferably from 50 to 500 nm, and more preferably from 50 to 300 nm.

Methods established in the prior art, such as extrusion through polycarbonate membranes, ethanol injection or high pressure homogenization, are used to produce the liposomes.

Methods of entrapping water-soluble active substances in liposomes are well-known to those skilled in the art. For inclusion of a desired active substance in liposomes, the active substance is dissolved in a buffer solution which is subsequently used to produce the liposomes. In the so-called passive inclusion, the relative volume enclosed by the liposomes being formed is an important issue. In passive inclusion, the inclusion efficiency is increased with increasing lipid concentration because the liquid volume enclosed by the lipid double layer is increased.

Passive inclusion is preferably used in those cases where large amounts of a readily soluble active substance are to be entrapped. To this end, liposomes with a lipid concentration of from 30 to 150 mM, preferably with a lipid concentration of from 50 to 120 mM, and more preferably with a lipid concentration of from 80 to 110 mM are produced in the presence of dissolved active substance.

Another method of entrapping water-soluble active substances is the so-called “advanced loading” method described in WO 01/34115 A2 which hereby is incorporated in the disclosure of the present invention. To achieve high inclusion efficiency, the active substance in another embodiment of the invention is entrapped in the liposomes using the advanced loading method. This method is preferably used in those cases where the active substance is to be enclosed in the liposomes as efficiently as possible and thus e.g. in a cost-saving manner. This method, which is based on the interaction between the active substance and membrane-forming substances, operates at low ionic strength and at a pH value where the active substance is present in a state of cationic charge so as to undergo reversible electrostatic interaction with the anionic liposomal membrane.

The charge of the active substances at a given pH can be inferred from data bases, such as SWISS-PROT, or can be estimated using well-known algorithms.

In another embodiment of the invention the passive inclusion method is combined with the advanced loading process. In this procedure, the advanced loading process is performed using a lipid concentration of from 30 to 150 mM, preferably a lipid concentration of from 50 to 120 mM, and more preferably a lipid concentration of from 80 to 110 mM, in order to significantly increase the inclusion rates compared to the separate methods.

Following liposome preparation, active substance adhering on the outside of the liposomal membrane can be detached and removed from the surface of the liposomes. This step is of crucial importance to the properties of the liposomal depot. Detaching the active substance from the liposome surface and removing it from the liposome suspension affords depot formulations having virtually no or only minimal “burst release”. In particular, this feature is of crucial importance in those cases where active substances are to be administered which may give rise to toxic reactions in the body even during a briefly high concentration of active substance, as is the case during initial arrival. One example for this is insulin, overdosage of which may give rise to live-threatening hypoglycemic conditions.

Termination of the existing interaction can be effected e.g. by changing the pH value or increasing the ionic strength. Final removal can be effected using methods well-known to those skilled in the art, such as centrifugation, ultrafiltration, dialysis, or other chromatographic methods, so that at least 90% of the active substance is entrapped in the liposome and less than 10%, preferably less than 5% of the active substance is outside the liposome.

In another embodiment of the invention the active substance adhering to the liposomal membrane is not detached from the membrane, i.e., the pH value or ionic strength remains unchanged. In particular, this embodiment finds use with active substances where initial arrival of the active substance is toxicologically safe, as is the case e.g. with leuprolide acetate or many antibodies. This embodiment also applies in those cases where the active substance is detached from the membrane, as described above, but not removed.

All or part of the free active substance, but more than 5%, preferably more than 10%, remains in the liposome suspension, providing for rapid initial arrival of active substance in the blood.

Another advantage of this embodiment is that the suspension can be lyophilized because, having equal concentrations of active substance on both the inner and outer surface of the membrane, release of active substance entrapped inside is minimized during the lyophilization process.

The anionic active substance-containing liposomes produced in this way are subsequently used to from the aggregates, to which end they are contacted with a polycation. More specifically, suitable polycations are chitosan, poly(dimethyldiallylammonium chloride), polyallylamine, polyethyleneimine, poly(dimethylaminoethyl acrylate), polylysine, polyhistidine, polyornithine, polyarginine, polyquats (starch derivatives with amino or ammonium groups), as well as copolymers thereof.

Surprisingly, it was found that cationic liposomes can also be used in the formation of such aggregates. To this end, stable cationic liposomes are employed which scarcely undergo fusion with anionic liposomes. Such liposomes include cationic lipids such as:
DAC-Chol 3-β-[N-(N′,N′-dimethylaminoethane)carbamoyl]-cholesterol
DC-Chol 3-β-[N-(N′,N′-dimethylaminoethane)carbamoyl]-cholesterol,
TC-Chol 3-β-[N-(N′,N′,N′-trimethylaminoethane)carbamo-yl]cholesterol,
BGSC Bis-guanidinium-spermidine-cholesterol,
BGTC Bis-guanidinium-tren-cholesterol,
DOTAP (1,2-dioleoyloxypropyl)-N,N,N-trimethylammonium chloride,
DOSPER (1,3-dioleoyloxy-2-(6-carboxyspermyl)propylamide),
DOTMA (1,2-dioleyloxypropyl)-N,N,N-trimethylammonium chloride (Lipofectin®),
DORIE (1,2-dioleyloxypropyl)-3-dimethylhydroxyethyl-ammonium bromide,
DOSC (1,2-dioleoyl-3-succinyl-sn-glycero choline ester),
DOGSDSO (1,2-dioleoyl-sn-glycero-3-succinyl-2-hydroxy-ethyl disulfide ornithine),
DDAB dimethyldioctadecylammonium bromide,
DOGS ((C18)₂GlySper3⁺) N,N-dioctadecylamido-glycyl-spermine (Transfectam®),

(C18)₂Gly⁺ N,N-dioctadecylamidoglycine,

DOEPC 1,2-dioleoyl-sn-glycero-3-ethylphosphocholine

or other O-alkylphosphatidyl cholines or ethanolamines,

1,3-bis(1,2-bis-tetradecyloxy-propyl-3-dimethylethoxyammonium bromide)-propan-2-ol (Neophectin®),

and the saturated derivatives with dimyristoyl, dipalmitoyl or distearoyl chains of all above-mentioned lipids with unsaturated fatty acid and/or fatty alcohol chains.

Preferred cationic lipids used in the practice of the invention comprise cholesteryl-3β-N-(dimethylaminoethyl) carbamate (DC-Chol), 3-β-[N-(N,N′-dimethylaminoethane)carbamoyl]cholesterol (DAC-Chol), (N-[1-(2,3-dimyristoyloxy)propyl]-N,N,N-trimethylammonium salt (DMTAP), (N-(1-(2,3-dipalmitoyloxy)propyl]-N,N,N-trimethylammonium salt (DPTAP), (N-[1-(2,3-dioleoyloxy)propyl]-N,N,N-trimethylammonium salt (DOTAP).

In addition to the cationic lipid itself, the cationic liposomes include particularly cholesterol and saturated neutral phosphatidyl cholines, preferably DPPC or DSPC.

In a particularly preferred fashion the level of cholesterol is between 35 and 50 mole-%, and the cationic lipids are preferably employed in the mixture with 5 to 20 mole-%.

Active substance-containing anionic liposomes and the polycation used in aggregation are preferably combined at a ratio of from 5:1 to 1:5, and these figures relate to the molar ratio of the charge carriers. Mixtures around the equivalence point, between a 2:1 and 1:2 level of the components, are particularly preferred.

The aggregates rapidly form upon simple mixing of the liposomal suspension with the polycation or the cationic liposomes. It is therefore possible to produce and store the two suspensions separately and combine them immediately prior to use. This can be done by simple mixing prior to injection. In a particularly preferred embodiment the two components are supplied in a double-chamber syringe and mixed by injecting.

However, industrial production of the aggregates is also possible, and these structures can be provided with a long shelf-life using lyophilization, for example.

In a particularly preferred and controlled fashion the production of such aggregates is effected in a continuous-flow reactor, such as described in WO 01/64330. Said printed document discloses a device allowing continuous coating of liposomes with polyelectrolytes.

The inventive teaching of aggregate formation is particularly good to control in such a machine. One special advantage of the method disclosed herein lies in the production of larger structures obtained by combining solutions (active substance liposomes and polycation) allowing sterile filtration beforehand. In this way, high regulatory requirements can be met.

The inventive liposomal aggregates containing active substances can be injected subcutaneously or intramuscularly as a depot medicinal form.

Furthermore, they can also be applied locally or topically, e.g. in the dressing of wounds or in cancer therapy. The depot systems according to the invention can also be used to accelerate the healing process or in postoperative care.

Accordingly, the invention relates to (a) a depot system, particularly for delayed release of active substances, comprising anionic liposomes comprising (i) saturated synthetic phosphatidyl cholines selected from the group comprising DMPC, DPPC and/or DSPC, (ii) cholesterol at a level of from 35 to 50 mole-%, (iii) anionic lipids selected from the group comprising DMPG, DPPG, DSPG, DMPS, DPPS and/or CHEMS at a level of from 5 to 20 mole-% in the liposomal membrane, (iv) at least one protein and/or peptide active substance, and (v) a cationic polymer, and (b) the use of said depot system preferably in in vivo systems, said depot system preferably having a depot time of at least one week, preferably more than 10 to 14 days, and more preferably longer than 3 weeks. Of course, it may also be preferred in particular uses that the depot time be less than one week, e.g. two, three, preferably four, more preferably five, or especially preferably six days.

The invention also relates to a kit comprising the depot system according to the invention, optionally together with information concerning combining the contents of the kit. The kit can be used in basic research and medicine. For example, the information can also be a reference to an internet address where further information can be obtained. The information can be a treatment regimen for a disease or e.g. instructions of how to use the kit in research.

Without intending to be limiting, the invention will now be explained in more detail with reference to the following examples.

Abbreviations

DMPC dimyristoylphosphatidyl choline

DPPC dipalmitoylphosphatidyl choline

DSPC distearoylphosphatidyl choline

DMPG dimyristoylphosphatidyl glycerol

DPPG dipalmitoylphosphatidyl glycerol

DSPG distearoylphosphatidyl glycerol

DMPS dimyristoylphosphatidyl serine

DPPS dipalmitoylphosphatidyl serine

DSPS distearoylphosphatidyl serine

DMPA dimyristoylphosphatidic acid

DPPA dipalmitoylphosphatidic acid

CHEMS cholesterol hemisuccinate

### EXAMPLE 1

Inclusion of Insulin in Liposomes

A mixture of 50 mole-% DPPC, 10 mole-% DPPG and 40 mole-% Chol is dissolved in chloroform at 50° C. and subsequently dried completely in vacuum in a rotary evaporator. The lipid film is added with human insulin solution (recombinant insulin; 7.5 mg/ml insulin in 10 mM glycine-HCl, 300 mM sucrose, pH 3) in an amount so as to form a 50 mM suspension. Subsequently, this suspension is hydrated in a water bath at 50° C. for 45 minutes by agitating and treated in an ultrasonic bath for another 5 minutes. Thereafter, the suspension is frozen. This is followed by 3 cycles of freezing and thawing, each thawing being followed by a 5 minute treatment in the ultrasonic bath.

Following final thawing, the liposomes are subjected to multiple extrusions through a membrane having a pore width of 200 nm or 400 nm (Avestin LiposoFast, polycarbonate membrane with a pore width of 200 or 400 nm). Following extrusion, the resulting suspension is rebuffered by adding a solutions of HEPES, pH 7.5, and NaCl. After filtration of the liposomes through 0.8 pm, non-entrapped insulin is removed by gel filtration (S-200 column, Pharmacia) . Following liberation from the liposomes, the amount of entrapped insulin is determined by means of an ELISA (DRG-ELISA Kit). Inclusion rates of 50-70% insulin are found.

### EXAMPLE 2

Inclusion of a Radiolabelled Model Cargo in Liposomes

A mixture of 50 mole-% DPPC, 10 mole-% DPPG and 40 mole-% Chol was dissolved in chloroform at 50° C. and subsequently dried completely in vacuum in a rotary evaporator. The lipid film is added with ³H-inulin solution (18.5 MBq/ml ³H-inulin in 10 mM HEPES, 150 mM NaCl, pH 7.5) in an amount so as to form a 100 mM suspension. Subsequently, this suspension is hydrated in a water bath at 50° C. for 45 minutes by agitating. Thereafter, the suspension is frozen.

Following thawing, the liposomes are subjected to multiple extrusions through a membrane having a pore width of 50, 200 or 400 nm (Avestin LiposoFast, polycarbonate membrane with a pore width of 50 nm, 200 nm or 400 nm). Removal of non-entrapped ³H-inulin is effected via gel filtration (G75 column Pharmacia) . Following removal, the amount of entrapped ³H-inulin is determined in a scintillation counter. Inclusion rates of 20-30% 3H-inulin are found.

In analogy, ³H-inulin-filled liposomes having the composition 60 mole-% DPPC, 10 mole-% DC-Chol and 30 mole-% Chol are produced (extrusion through 200 nm).

### EXAMPLE 3

Aggregation of Negatively Charged Liposomes with Positively Charged Polymers and Positively Charged Liposomes

Following gel filtration, 2 ml of each of the liposomes obtained in Example 2, having the composition 50 mole-% DPPC, 10 mole-% DPPG and 40 mole-% Chol (extrusion through 100 or 200 nm), are rapidly mixed in a tube with a solution of the polymers PLL or chitosan or with cationic blank liposomes having the composition 60 mole-% DPPC, 10 mole-% DC-Chol and 30 mole-% Chol:

TABLE 1
Aggregation of negative liposomes with various
positive polymers or liposomes

| | Polymer/liposome | pH value of |
|---|---|---|
| Formulation/concentration | amount | aggregation |
| DPPC/DPPG/Chol 50:10:40 (200 nm) | Chitosan | 5 |
| 12.5 mM | 2.5 mg in 100 μl | |
| DPPC/DPPG/Chol 50:10:40 (200 nm) | Poly-L-lysine | 7.5 |
| 12.5 mM | 0.625 mg in 100 μl | |
| DPPC/DPPG/Chol 50:10:40 (200 nm) | DC-Chol liposomes | 7.5 |
| 12.5 mM | (100 mM) | |
| | 250 μl | |
| DPPC/DPPG/Chol 50:10:40 (100 nm) | Poly-L-lysine | 7.5 |
| 12.5 mM | 0.625 mg in 100 μl | |

### EXAMPLE 4

Use of Liposomal Depot Systems in an Animal Model

Negatively charged liposomes (³H-inulin cargo) aggregated with positive polymers or liposomes (cf. Examples 2 and 3) were injected subcutaneously in healthy rats at a concentration of 12.5 mM lipid in a volume of 0.5 ml. A control sample with blank liposomes and ³H-inulin was likewise administered subcutaneously in a volume of 0.5 ml. The pharmacokinetic data were obtained by blood sampling at varying points in time and subsequent scintillation measurements. The test period of the animal study was 2 weeks in total. According to the evaluation, there were only two formulations where the animals showed slight local adverse reactions (reddening at the point of injection) which, however, had healed after 10 days at latest. The general condition of all animals was good over the test period. The formulations and relative bioavailabilities up to t=336 h are illustrated in the following table:

| | | Relative bioavailability |
|---|---|---|
| Formulation | Composition | up to t = 336 h [%] |
| K 2 | DPPC/DPPG/Chol | 100 |
| | 50:10:40 (200 nm) + ³H-inulin | |
| | outside | |
| P12 | DPPC/DPPG/Chol | 27.03 |
| | 50:10:40 (200 nm) + chitosan | |
| P13 | DPPC/DPPG/Chol | 23.45 |
| | 50:10:40 (200 nm) + PLL | |
| P14 | DPPC/DPPG/Chol | 28.4 |
| | 50:10:40 (200 nm) + DC- | |
| | Chol liposomes | |
| P19 | DPPC/DPPG/Chol | 34.45 |
| | 50:10:40 (100 nm) + PLL | |

### EXAMPLE 5

Inclusion of Leuprolide Acetate in Liposomes

A lipid mixture having the following composition

| Formulation | Composition |
|---|---|
| L1 | DPPC/DPPG/Chol |
| | 60:10:40 (mole-%) |

is dissolved in chloroform at 50° C. and subsequently dried completely in vacuum in a rotary evaporator. The lipid film is added with leuprolide acetate solution (2.5 mg/ml in 10 mM HEPES, 150 mM NaCl, pH 6) in an amount so as to form a 100 mM suspension. Subsequently, this suspension is hydrated in a water bath at 50° C for 45 minutes with agitating. Thereafter, the suspension is frozen. This is followed by 3 additional cycles of freezing and thawing.

Following final thawing, the liposomes are subjected to multiple extrusions through a membrane having a pore width of 400 nm (Avestin LiposoFast, polycarbonate membrane with a pore width of 400 nm).

Following removal of free leuprolide acetate, the percentage of entrapped leuprolide acetate is determined by means of triple sedimentation in an ultracentrifuge at 60,000×g for 45 minutes. The leuprolide acetate is determined following extraction with CHCl₃ and CH₃OH, using RP-HPLC. Inclusion rates of about 15% leuprolide acetate are found.

Non-entrapped leuprolide acetate is not removed in the samples used in the animal experiments. 2 ml of the liposomes containing leuprolide (12.5 mM in lipid) are rapidly mixed in a tube with 250 μl of blank cationic liposomes having the composition 60 mole-% DPPC, 10 mole-% DC-Chol and 30 mole-% Chol (100 mM in lipid).

### EXAMPLE 6

Use of the Liposomal Depot System in an Animal Model

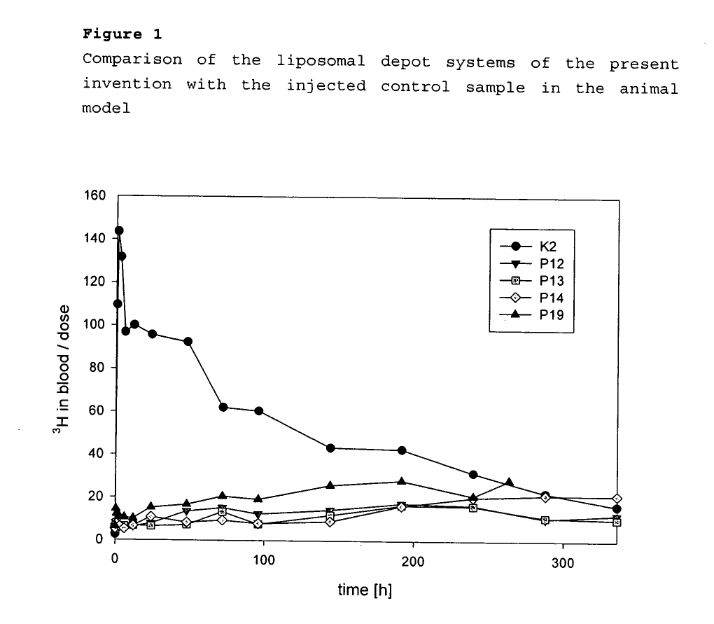

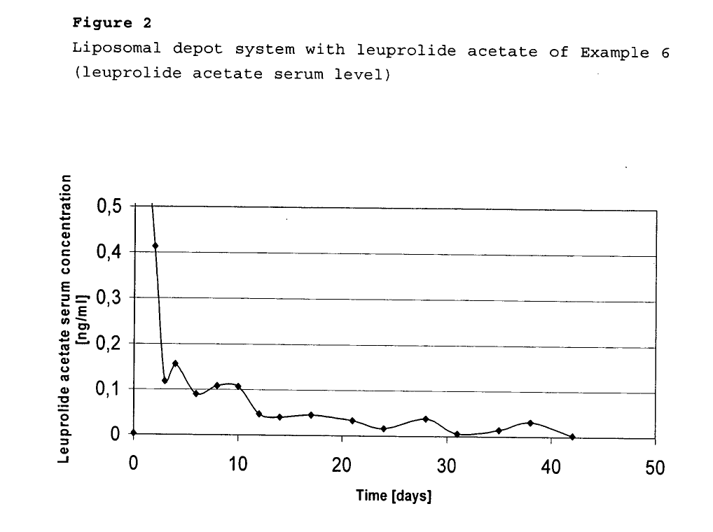

Without removing the active substance present outside, the liposomal aggregates of Example 5 were injected subcutaneously in healthy male rats (3 animals) in a volume of 0.5 ml. The leuprolide acetate dose per animal was 2.5 mg. The pharmacokinetic data were obtained by blood sampling at varying points in time, obtaining serum and determining the leuprolide acetate concentration in the serum by means of ELISA (Peninsula). The test period of the animal study was 6 weeks in total. The general condition of all animals was good over the test period. The pharmacokinetic data are shown in FIG. 2.

1. A depot system for a delayed release of active substances, the depot system comprising anionic liposomes, the anionic liposomes—comprising:
saturated synthetic phosphatidyl cholines selected from one or more from the group consisting of DMPC, DPPC and DSPC,
cholesterol at a level ranging from 35 to 50 mole-%,
anionic lipids selected from one or more from the group consisting of DMPG, DPPG, DSPG, DMPS, DPPS and CHEMS at a level ranging from 5 to 20 mole-% in the liposomal membrane, and
the depot system further comprising:
at least one from the group consisting of protein and peptide active substance, and
a cationic polymer or cationic liposome,

2. The depot system according to claim 1, wherein the cationic polymer is selected from the group consisitng of chitosan, poly(dimethyldiallylammonium chloride), polyallylamine, polyethyleneimine, poly(dimethylaminoethyl acrylate), polylysine, polyhistidine, polyomithine, polyarginine, polyquats and copolymers thereof.

3. The depot system according to claim 1, wherein at least 90% of the active substance is enclosed in the liposome and less than 10% is outside the liposome.

4. The depot system according to claim 1, wherein the active substance is entrapped in the liposome and more than 10% thereof is outside the liposome.

5. (canceled)

6. The depot system according to claim 1, wherein the cationic liposomes comprise
5 to 20 mole-% of cationic lipid,
35 to 50 mole-% of cholesterol, and
saturated phosphatidyl cholines.

7. The depot system according to claim 6, wherein the cationic lipids are selected from at least one from the group consisting of DAC-Chol, DC-Chol, DMTAP, DPTAP and DOTAP.

8. The depot system according to claims 1, wherein the anionic liposomes are mixed with the cationic polymers or with the cationic liposomes at a charge carrier molar ratio ranging from 5:1 to 1:5.

9. The depot system according to claims 1, wherein the depot system is capable of delivery of the active substance for at least 1 week.

10. The depot system according to claim 1, wherein the size of the anionic liposomes varies from 20 to 1,000 nm.

11. The depot system according to claim 9, said active substance comprising a water-soluble active substance derivative selected from the classes of active substances consisting of antibiotic, antimycotic, cytostatic agents and glucocorticoids.

12. The depot system according to claim 1, wherein said liposomes comprise one or more from the group consisting of LHRH agonists and GnRH analogs, as active substance.

13. The depot system according to claim 1, wherein the active substance comprises insulin.

14. The depot system according to claim 1, wherein said active substance comprises heparin.

15. The depot system according to claim 1, wherein said active substance comprises antigen fragments for vaccination.

16.-19. (canceled)

20. The depot system according to claim 6, wherein the saturated phosphatidyl cholines are chosen from the group consisting of DPPC and DSPC.

21. The depot system according claim 1, wherein the anionic liposomes are mixed with the cationic polymers or with the cationic liposomes at a charge carrier molar ratio ranging from 2:1 to 1:2.

22. The depot system according claim 1, wherein the size of the anionic liposomes varies from 50 to 800 nm.

23. The depot system according to claim 1, wherein the size of the anionic liposomes varies from 50 to 300 nm.

24. The depot system according to claim 12, wherein said liposomes comprise one or more from the group consisting of leuprolide acetate, buserelin, goserelin and triptorelin as active substance.

25. A drug comprising a depot system according to claim 1.

26. Method of administering the depot system according to claim 1, comprising the step of injecting the depot system subcutaneously or intramuscularly.

27. Method of administering the depot system according to claim 1, comprising the step of one or both from the group consisting of topical and local application to support healing processes.

28. Method of treatment, comprising the step of using a depot system according to claim 1 to support wound healing.
